(19) United States
(12) Patent Application Publication
(10) Pub. No.: US 2026/0102219 A1
(43) Pub. Date: Apr. 16, 2026

(54) Cover Assembly for Medical Device with Replaceable Attachment

(71) Applicant: CIVCO Medical Instruments Co., Inc., Kalona, IA (US)

(72) Inventor: Craig J. Cermak, Iowa City, IA (US)

(21) Appl. No.: 19/358,178

(22) Filed: Oct. 14, 2025

Related U.S. Application Data
(60) Provisional application No. 63/707,418, filed on Oct. 15, 2024.

(51) Int. Cl. A61B 50/00

(52) U.S. Cl. CPC A61B 50/00

(57) ABSTRACT

A cover assembly configured to cover at least a portion of a medical device, wherein the medical device has a body and an attachment removably coupled to the body at an attachment plate positioned at a distal end of the body. The cover assembly includes a film material forming a tubular sheath. The tubular sheath defines a passage along a longitudinal axis of the tubular sheath between a first end and an opposing second end. The tubular sheath includes a single layer wall portion at the second end of the tubular sheath. The single layer wall portion transitions into a double layer wall portion at a first fold area. The double layer wall portion defines a throttle area configured to retain the body of the medical device at the attachment plate. Also disclosed are example methods for producing the cover assembly.

### CROSS-REFERENCE TO RELATED APPLICATIONS

This application claims the benefit of U.S. Provisional Patent Application No. 63/707,418 filed October 15, 2024, the entire contents of which is hereby incorporated by reference herein.

### BACKGROUND

Conventional covers for covering a medical device during a surgical procedure to prevent or limit contamination in a sterile environment typically include a thin film tube defining a lumen or a passage in which a medical device is positioned. An initially closed distal end of the tube includes a line of perforation, allowing the surgeon to remove a portion of the tube distal to the line of perforation to provide an opening in communication with the lumen or passage. With the medical device positioned within the tube, the distal end of the handle of the medical device fits within the opening formed in the distal end of the tube.

During surgical procedures, it is difficult for the surgeon to remove a connector or an attachment, for example, from the handle and/or to attach a new, sterile connector or attachment to the distal end of the handle because the surgeon is required to open the distal end of the cover with one hand to access the connector or attachment positioned within the passage and remove the connector or attachment with the other hand.

### BRIEF DESCRIPTION OF THE DRAWINGS

FIG. 1 is an example medical device for which the example cover assembly, according to example embodiments, is configured to protect from undesirable contamination;

FIG. 2 is a perspective view of a portion of an example cover assembly covering a portion of a compatible medical device, according to an example embodiment;

FIG. 3 is a side view of a portion of the example cover assembly shown in FIG. 2;

FIG. 4 is a perspective view of a portion of the example cover assembly shown in FIG. 2;

FIG. 5 is another perspective view of a portion of the example cover assembly shown in FIG. 2;

FIG. 6 is another perspective view of a portion of the example cover assembly shown in FIG. 2;

FIG. 7 is a cross-sectional view of a portion of the example cover assembly shown in FIG. 2;

FIG. 8 is a side view of a portion of an alternative example cover assembly, according to an example embodiment;

FIG. 9 is a side view of a portion of the example cover assembly shown in FIG. 7 covering a portion of a compatible medical device, according to an example embodiment;

FIG. 10 is a side view of a portion of an alternative example cover assembly, according to an example embodiment;

FIG. 11 is a side view of a portion of the example cover assembly shown in FIG. 9 covering a portion of a compatible medical device, according to an example embodiment;

FIG. 12 is a side view of a portion of an alternative example cover assembly, according to an example embodiment;

FIG. 13 is a side view of a portion of the example cover assembly shown in FIG. 11 covering a portion of a compatible medical device, according to an example embodiment;

FIG. 14 is a side view of a portion of an alternative example cover assembly, according to an example embodiment;

FIG. 15 is a side view of a portion of the example cover assembly shown in FIG. 13 covering a portion of a compatible medical device, according to an example embodiment;

FIG. 16 is a side view of a portion of an alternative example cover assembly, according to an example embodiment;

FIG. 17 is a side view of a portion of the example cover assembly shown in FIG. 15 covering a portion of a compatible medical device, according to an example embodiment;

FIG. 18 is a side view of a portion of an alternative example cover assembly, according to an example embodiment;

FIG. 19 is a side view of a portion of the example cover assembly shown in FIG. 17 covering a portion of a compatible medical device, according to an example embodiment;

FIG. 20 is a side view of a portion of an alternative example cover assembly, according to an example embodiment;

FIG. 21 is a side view of a portion of an alternative example cover assembly, according to an example embodiment;

FIG. 22 is a side view of a portion of an alternative example cover assembly, according to an example embodiment;

FIG. 23 is a flow chart of an example method of forming an example cover assembly, according to an example embodiment; and

FIG. 24 illustrates folding steps of the example method shown FIG. 23.

Corresponding reference numbers indicate corresponding parts throughout the drawings.

### DETAILED DESCRIPTION

Various example embodiments and/or implementations will be described in detail with reference to the accompanying drawings. The same reference numbers may be used throughout the drawings to refer to the same or like parts. References made throughout this disclosure relating to specific embodiments and implementations are provided solely for illustrative purposes but, unless indicated to the contrary, are not meant to limit all embodiments and/or implementations. The following detailed description of certain example embodiments or implementations will be better understood when read in conjunction with the appended drawings.

During surgical procedures, surgeons commonly utilize medical devices including, without limitation, ablation probes or catheter systems, that include removable attachments at a distal end of a body or a handle of the medical device. During such surgical procedures, an initial attachment can be decoupled (e.g., removed, detached, etc.) from the body of the medical device and a new, sterile attachment, the same or different from the initial attachment, can be coupled to the body. Aspects of the disclosure provide a relatively thin, flexible film material, such as a suitable polyethylene film material, that is formed into a tubular or cylindrical sheath or cover defining a passage suitable to accommodate at least a portion of the medical device that includes at least a portion of the body or the handle, an attachment positionable at a distal end of the body (e.g., removably coupled to an attachment plate at the distal end of the body), and a cord or cable coupled to an opposite proximal end of the body. In example embodiments, a first end (e.g., a distal end) of the example tubular sheath forms a throttle area (e.g., a two-dimensional hourglass-shaped throttle area) including a plurality of layers of the polyethylene film material. An opposite second end (e.g., a proximal end) of the tubular sheath is open to allow fluid communication with the passage extending between the second end and the opposite first end of the tubular sheath to facilitate insertion of the medical device into the passage. As used herein to describe example embodiments, the term “distal” refers to a position of a structure further away from its origin (e.g., a distal end of the tubular sheath) while the term “proximal” refers to a position of a structure closer to its origin (e.g., a proximal end of the tubular sheath). In alternative example embodiments, the first end refers to the proximal end of the tubular sheath, while the second end refers to the opposite distal end of the tubular sheath.

The throttle area allows the distal end of the tubular sheath to extend a desired length distally past the throttle area but within a sterile field provided by the tubular sheath to allow the surgeon to remove the used attachment and attach a new, sterile attachment to the body of the medical device to prevent or limit undesirable contamination of the medical device during the surgical procedure.

Aspects of the disclosure provide a flexible polyethylene film material formed into a tubular sheath that includes multiple film material layers at the distal end of the tubular sheath that are formed to facilitate the insertion, detachment, and/or connection of a disposable attachment (e.g., connector, coupler, fitting, adapter, accessory, component, etc.) to the distal end of the medical device. In example embodiments, a first portion of the tubular sheath is inverted and folded back onto itself on an exterior surface of the tubular sheath. The inversion extends from the distal end of the tubular sheath past the throttle area. In example embodiments, this inversion and folding increases a structural integrity of the tubular sheath such that the distal end of the cover assembly opens or extends radially outwardly when the body or the handle of the medical device is inserted through a proximal opening in the tubular sheath and positioned within the passage at the throttle area. Additionally, a first portion (e.g., a first lateral side) of the distal end of the tubular sheath is folded back a suitable distance (e.g., 0.5 centimeter (cm) to 2.0 cm, 0.5 cm to 1.0 cm, etc.) such that the first portion of the distal end of the tubular sheath is shorter than a second portion (e.g., a second lateral side opposite the first lateral side) of the distal end of the tubular sheath. These fold areas facilitate urging or biasing the distal end of the tubular sheath to open or extend radially outwardly at the distal opening, with the second portion longer than the first portion to facilitate inserting the attachment into the throttle area with the body or the handle positioned within the passage. Therefore, a surgeon can more easily access and remove an attachment during a surgical procedure.

Further aspects of the disclosure provide an example method for producing a cover assembly configured to surround or cover at least a portion of a medical device having a body (e.g., a handle) and an attachment (e.g., connector, coupler, fitting, adapter, accessory, component, etc.) removably coupled to the body at an attachment plate positioned at a distal end of the body. The example method includes cutting to a length a suitable film material forming a tubular sheath. The tubular sheath initially forms a single layer wall configured to define a passage along a longitudinal axis extending along a length of the tubular sheath between a first end (e.g., a distal end) and an opposing second end (e.g., a proximal end). The first end of the tubular sheath is folded outwardly (i.e., folded radially outwardly from the longitudinal axis) at a first fold line to form a first fold area including a double layer wall portion. Although the tubular sheath is described as being folded outwardly, in example embodiments, the tubular sheath may be folded inwardly (i.e., folded radially inward towards the longitudinal axis). In certain example embodiments, the method further includes folding, at a second fold line, at least a portion of the first end of the tubular sheath (e.g., a first lateral side portion) to form a second fold area comprising a quadruple layer wall portion at a second fold area adjacent to or within the first fold area. The double layer wall portion and the quadruple layer wall portion are sealed at the first fold area and the second fold area, respectively, to form a throttle area configured to properly (e.g., securely) retain the body of the medical device at the attachment plate within the passage of the tubular sheath and urge, or bias, the distal end of the tubular sheath to open or extend radially outwardly at a distal opening of the tubular sheath to facilitate inserting the attachment into the throttle area with the body positioned within the passage of the tubular sheath.

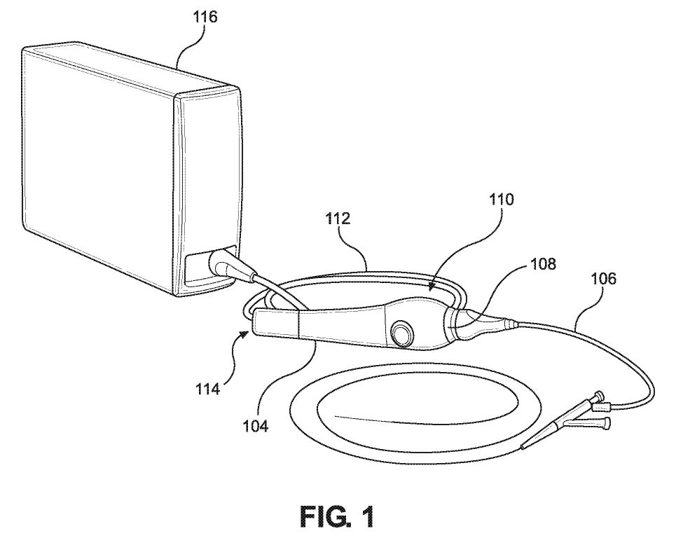

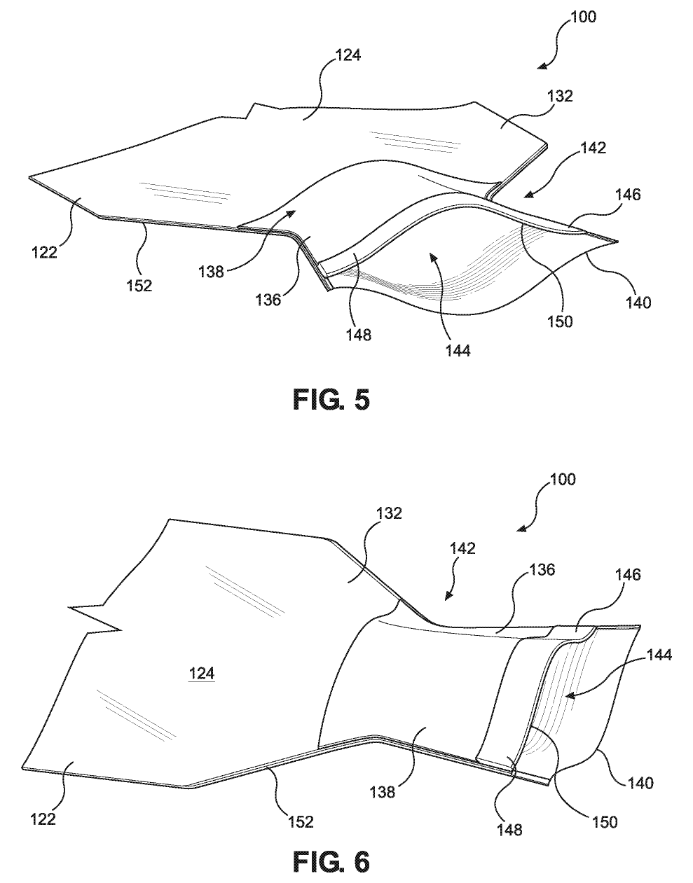

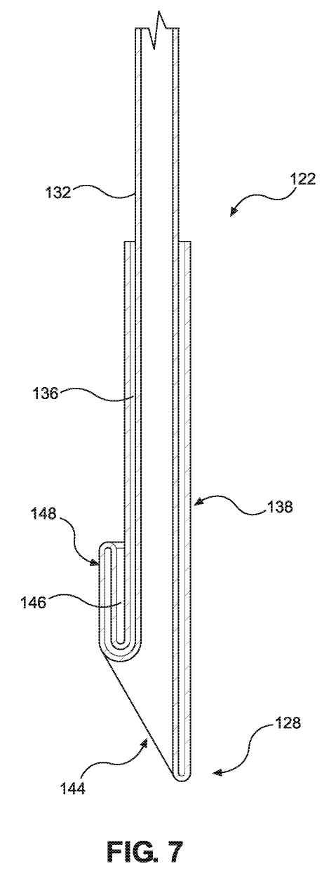

Referring initially to FIGS. 1-7, in example embodiments, a cover assembly 100 is configured to cover (e.g., surround, enclose, shield, etc.) at least a portion of a medical device 102, as shown in FIG. 1. In example embodiments, cover assembly 100 is configured to cover or surround at least a portion of any suitable medical device, including, without limitation, medical device 102 having a body 104 and an attachment 106, such as a removable interventional catheter shown in FIG. 1. Other suitable medical devices include an ablation probe, for example. Attachment 106 is removably coupled to body 104 at an attachment plate 108 positioned at a distal end 110 of body 104. Medical device 102 includes a power cord or connector cable 112 coupled (e.g., removably coupled) to a proximal end 114 of body 104. Connector cable 112 may be coupled to a power generator 116, as shown in FIG. 1, or directly into a power source or computer (not shown), for example. In example embodiments, cover assembly 100 includes a film material 120 formed from a film material stock into a tubular sheath 122. In certain example embodiments, film material 120 includes a suitable polyethylene material. In alternative example embodiments, film material 120 is formed of any suitable material for forming a relatively soft, flexible cover assembly that provides or promotes a sterile barrier including, without limitation, other suitable polymeric materials including, for example, an EMA copolymer, PMMA/polymethyl methacrylate (acrylic), amorphous polyethylene terephthalate (APT), blown polypropylene, cast polypropylene (CPP), fluoropolymers, high density polyethylene (HDPE), high performance film materials, high impact polystyrene (HIPS), low density polyethylene (LDPE), polybutylene terephthalate (PBT), polyethylene terephthalate (PET), polyethylene terephthalate glycol (PETG), polycarbonate, polypropylene (PP), polyphenylene ether (PPE), polyvinyl chloride (PVC), recycled polyethylene terephthalate (RPET), ultra-high molecular weight (UHMW) film materials, or urethane (polyurethane).

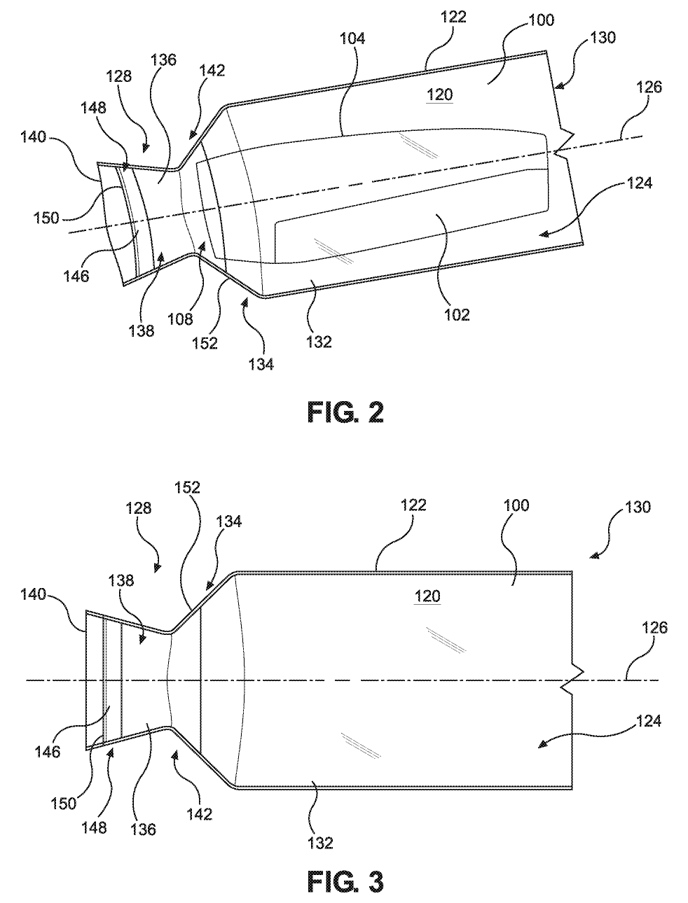

Tubular sheath 122 defines a passage 124 along a longitudinal axis 126 extending along a length of tubular sheath 122 between a distal end 128 and an opposing proximal end 130. Tubular sheath 122 is initially formed of a single layer wall portion 132 at proximal end 130 of tubular sheath 122. As shown in FIGS. 2-7, single layer wall portion 132 converges (e.g., tapers, narrows, etc.) toward longitudinal axis 126 at distal end portion 134 of single layer wall portion 132 and transitions into a double layer wall portion 136 at a first fold area 138. More specifically, in example embodiments, single layer wall portion 132 is folded at a first fold line 140 within first fold area 138 perpendicular to longitudinal axis 126. The first fold line 140 defines a distal peripheral edge of double layer wall portion 136 and tubular sheath 122. Single layer wall portion 132 folds over a portion of itself at first fold area 138 and forms double layer wall portion 136. Double layer wall portion 136 at least partially defines a throttle area 142 (e.g., throttle region, throttle section, neck, etc.) configured to retain body 104 of medical device 102 at attachment plate 108 within passage 124. Passage 124 is narrowed, or constricted, within the throttle area 142.

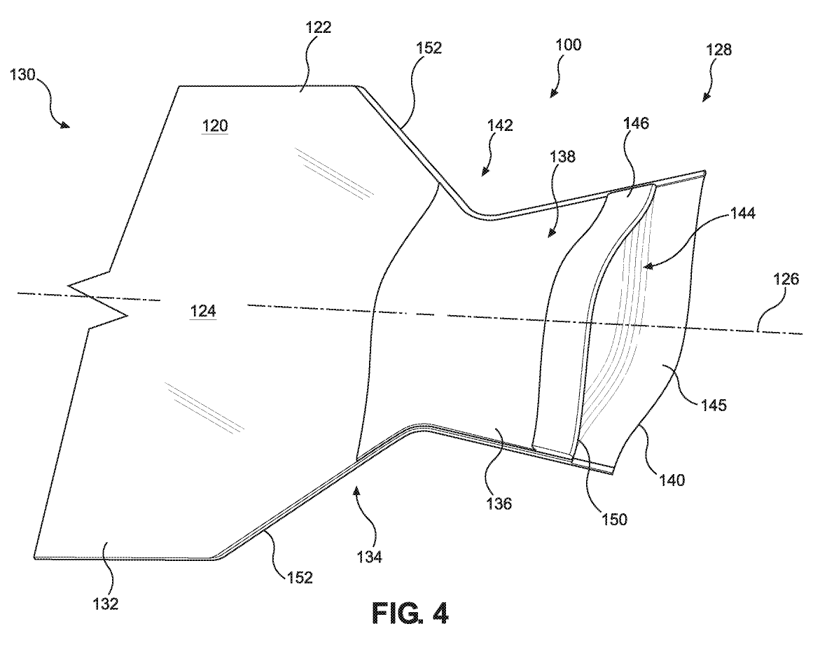

In example embodiments, double layer wall portion 136 converges toward longitudinal axis 126 within first fold area 138 and, more specifically, within throttle area 142, to form an opening 144 at distal end portion 134. In example embodiments, double layer wall portion 136 diverges away from longitudinal axis 126 (e.g., widens, flares outward, etc.) past the throttle area 142 and forms opening 144. Opening 144 is configured to receive attachment 106. Opening 144 is biased from a closed position to an expanded or open position and diverges or extends radially outwardly from longitudinal axis 126, as shown in FIGS. 4-6, for example. Opening 144, biased to an open position, thereby provides fluid communication with passage 124 to facilitate decoupling attachment 106 from body 104 and/or coupling attachment 106 to body 104. In example embodiments, opening 144 is configured to receive attachment 106 and other attachments different from attachment 106.

At throttle area 142 (e.g., at a proximal side of throttle area 142), double layer wall portion 136 is configured to retain body 104 at attachment plate 108 within a sterile field. For example, body 104 at attachment plate 108 can be maintained within a sterile field during a medical or surgical procedure. In certain example embodiments, at least a portion 145 (e.g., a lateral side portion) of double layer wall portion 136 extends distally from attachment plate 108 along a length of attachment 106 to further define, and extend, the sterile field.

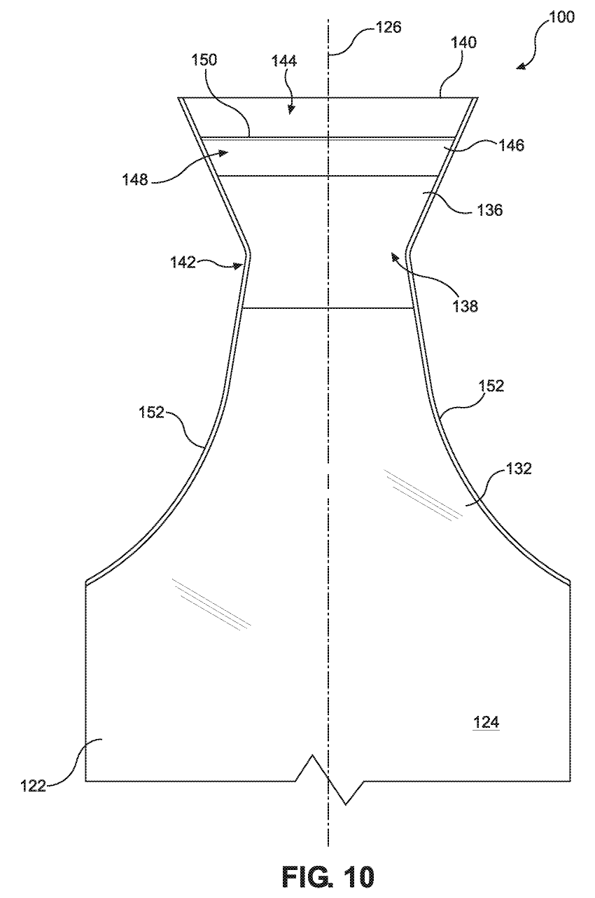

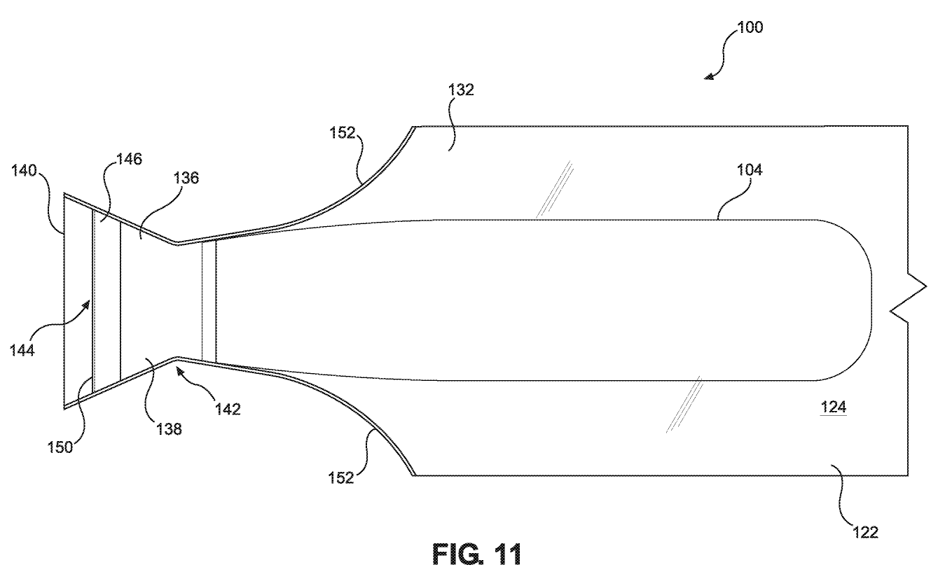

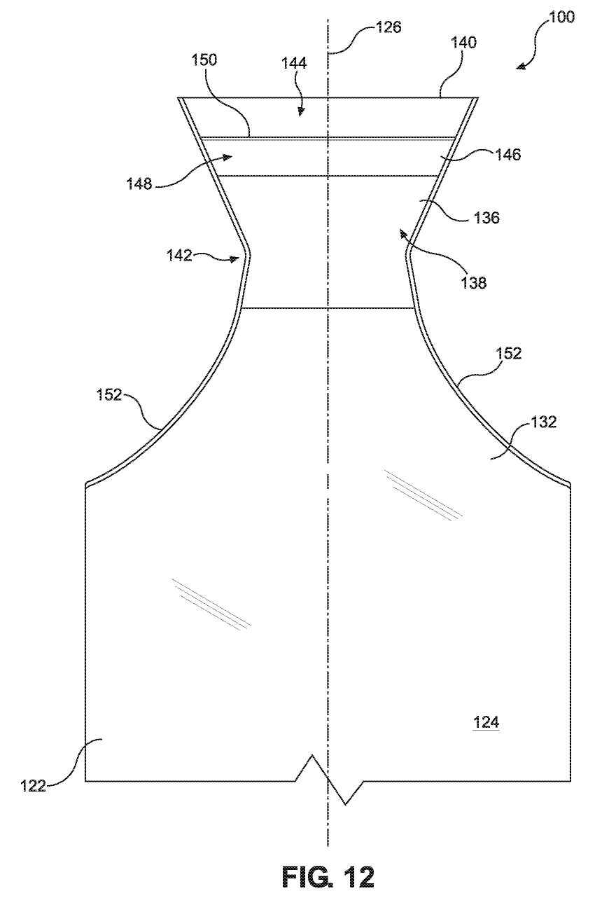

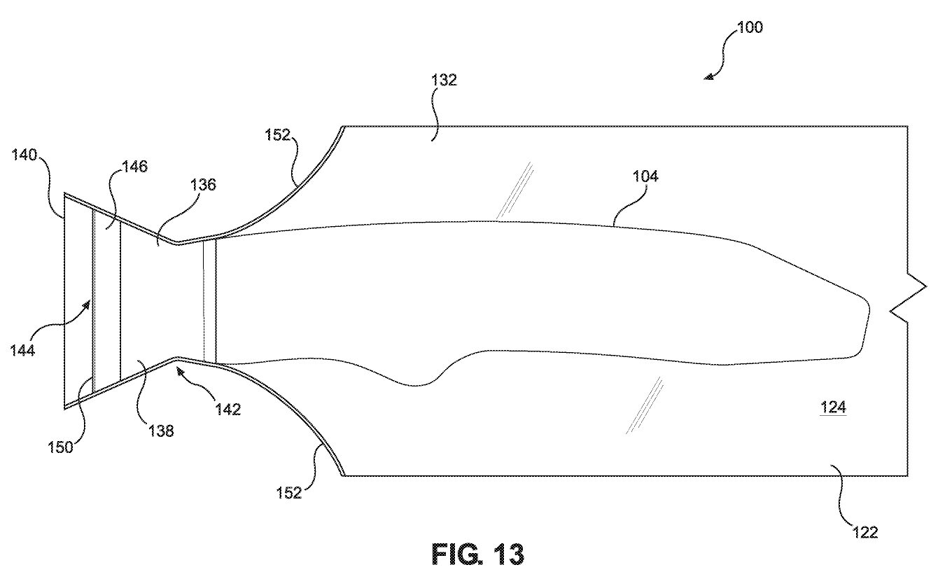

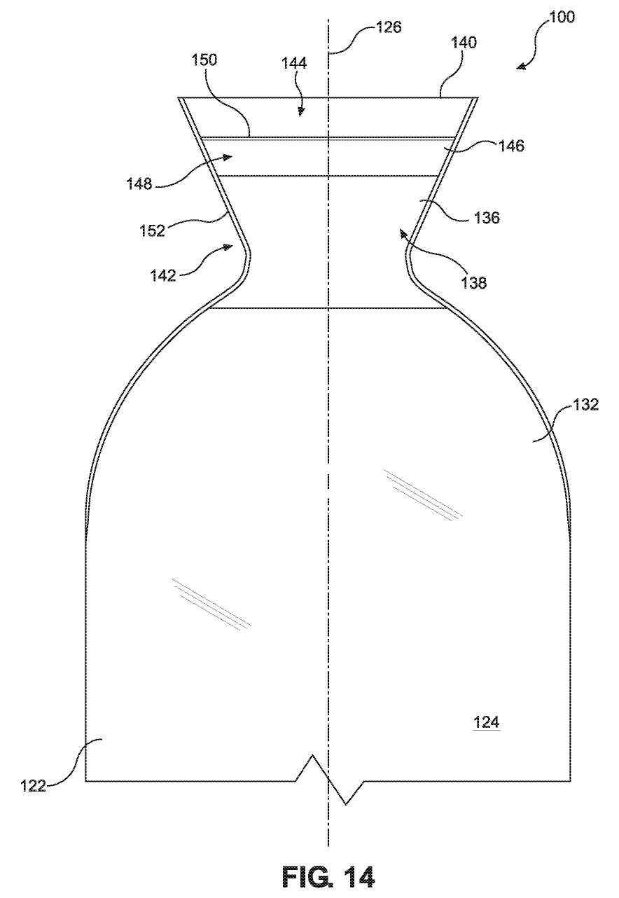

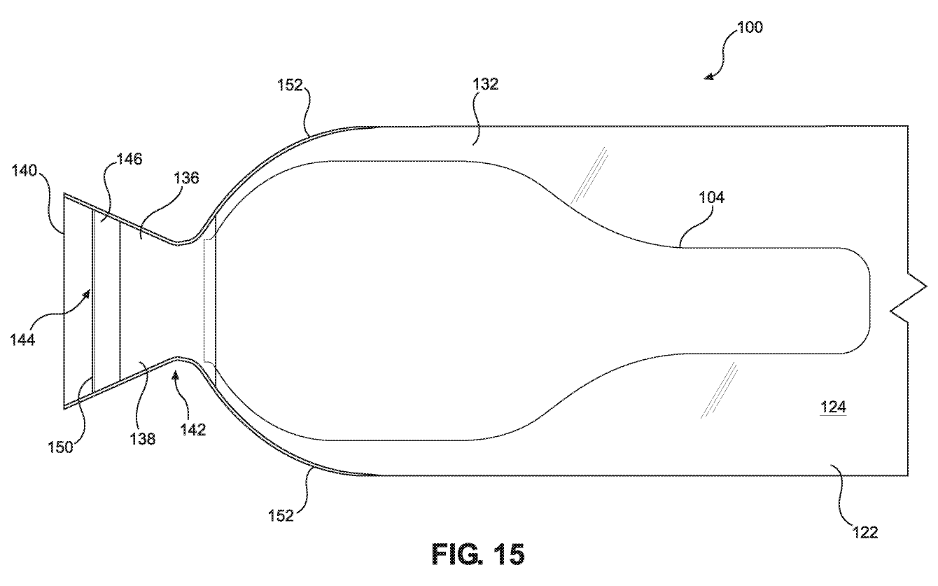

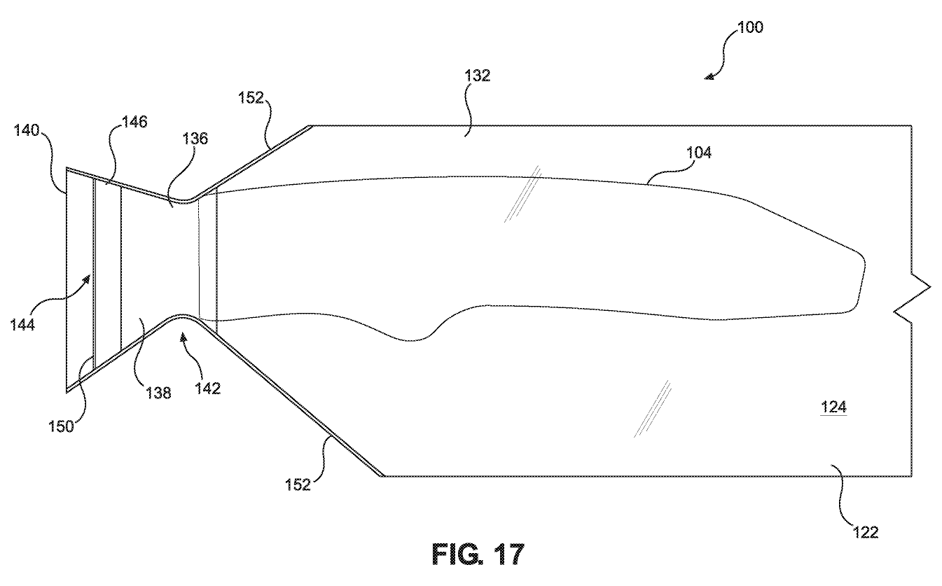

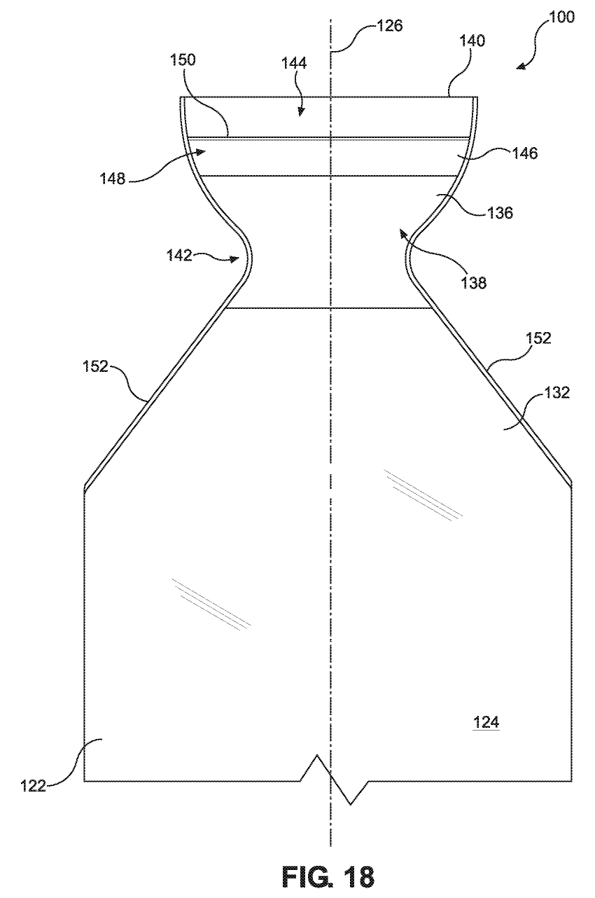

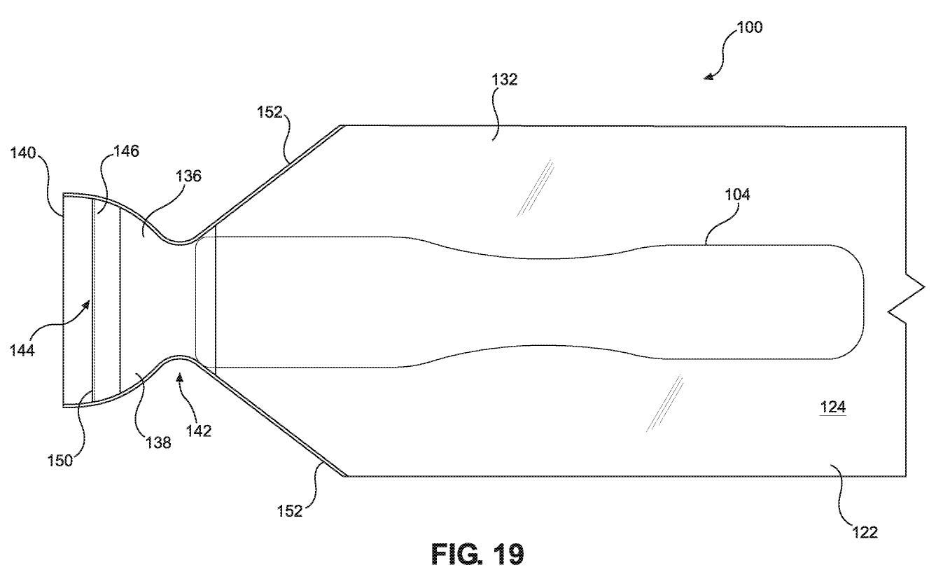

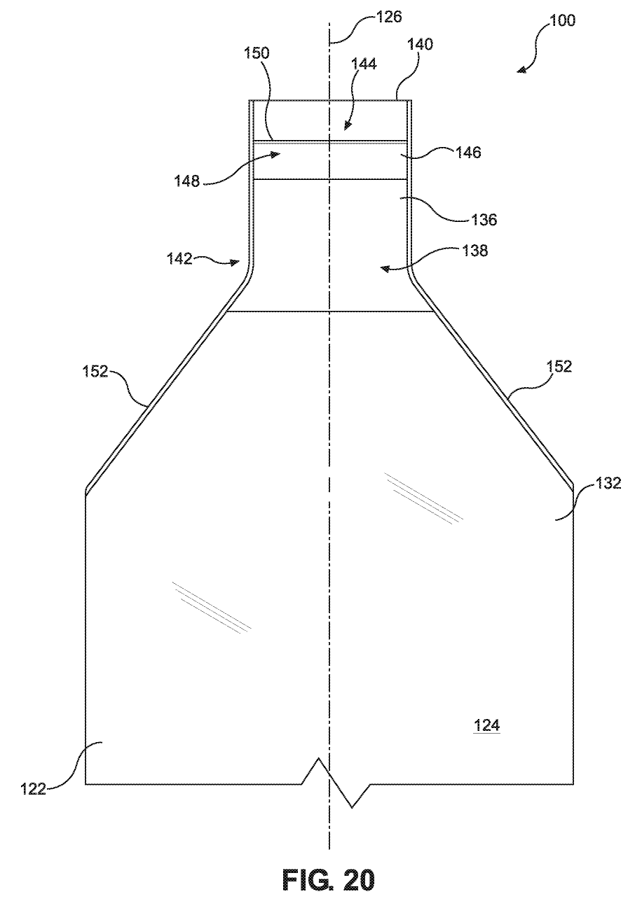

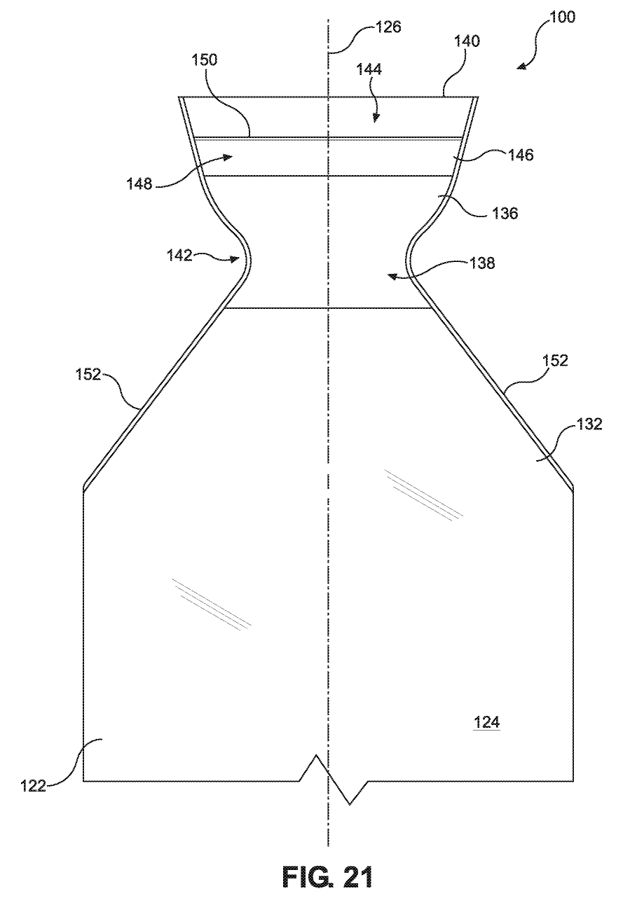

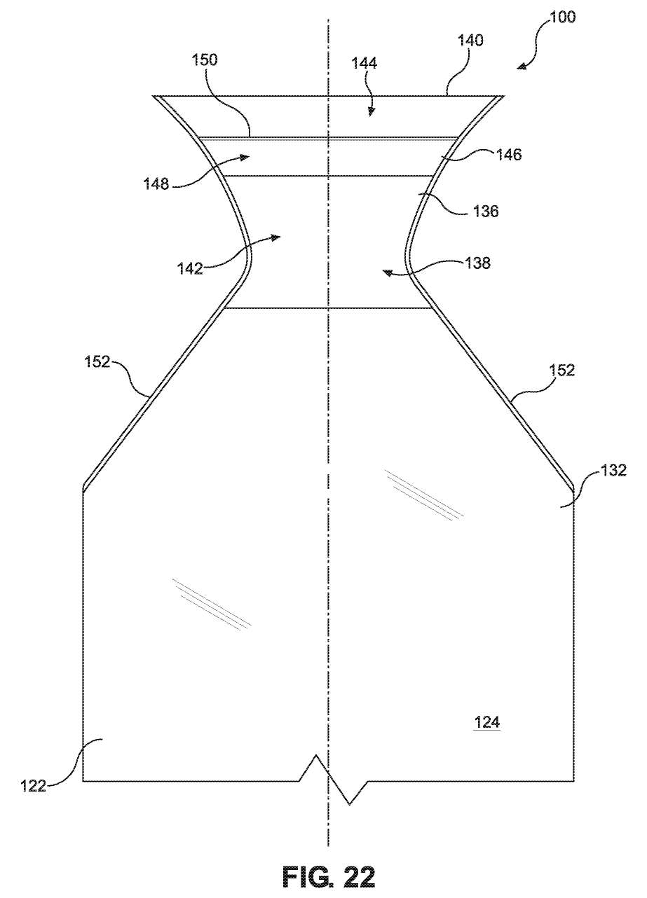

In certain example embodiments, double layer wall portion 136 transitions into a quadruple layer wall portion 146 at a second fold area 148 adjacent (e.g., proximal to) first fold area 138. More specifically, in example embodiments, double layer wall portion 136 transitions into quadruple layer wall portion 146 at a second fold line 150 extending perpendicular to longitudinal axis 126 (e.g., parallel to first fold line 140) and positioned proximally to first fold line 140. The second fold line 150 defines a distal peripheral edge of the quadruple layer wall portion 146. In example embodiments, second fold line 150 is stepped below, or offset from first fold line 140 such that at least a portion 145 of double layer wall portion 136 extends beyond the second fold line 150 and forms a lip, or flap. In example embodiments, distal end 128 of tubular sheath 122 is formed into throttle area 142. One or more of single layer wall portion 132, double layer wall portion 136, and quadruple layer wall portion 146 is sealed using a suitable sealing technique including, without limitation, a heat-sealing process (e.g., impulse heat sealing, radio frequency sealing, ultrasonic sealing, hot bar sealing, etc.), a thermoforming process, an adhesive process (e.g., adhesive bonding, etc.), and/or a chemical-sealing process (e.g., solvent bonding, etc.), to form throttle area 142, as shown, for example, in FIGS. 2-22. More specifically, after each of the first fold area 138 and second fold area 148 are formed, single layer wall portion 132, double layer wall portion 136, and quadruple layer wall portion 146 are sealed with a suitable seal 152 at lateral sides of tubular sheath 122, as shown in FIGS. 2-22.

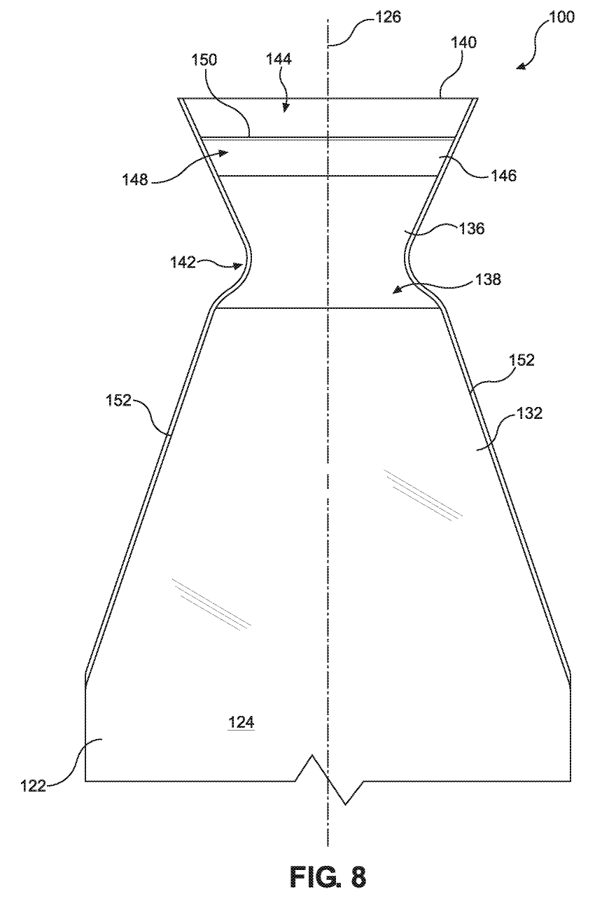

FIGS. 8-22 show example alternative embodiments of cover assembly 100 configured to cover or surround at least a portion of medical device 102 including, without limitation, medical device 102 having body 104 and attachment 106, such as a removable interventional catheter system or an ablation probe, for example, removably coupled to body 104 at attachment plate 108 positioned at distal end 110 of body 104. Example cover assemblies 100 shown in FIGS. 8-22 include a first portion (e.g., single layer wall portion 132) including a single layer film material forming passage 124 along longitudinal axis 126 of the first portion between a first end, e.g., a distal end, and a second end, e.g., a proximal end, of the first portion. In example embodiments, cover assembly 100 is formed of a polyethylene film material or other suitable film material known to those having ordinary skill in the medical device field.

A second portion ( e.g., double layer wall portion 136) is formed at the first end (e.g., a distal end) of the first portion. The second portion includes two layers of film material configured to cover or surround at least the distal end of body 104. In example embodiments, the second portion is formed by folding the first portion outwardly from the longitudinal axis and in a proximal direction along longitudinal axis 126 at first fold line 140. In certain example embodiments, a third portion (e.g., quadruple layer wall portion 146) is formed adjacent to or within the second portion. In example embodiments, the second portion is folded outwardly from longitudinal axis 126 and in a proximal direction along longitudinal axis 126 at second fold line 150 to form the third portion having four layers of film material. Although the first portion and the second portion are described as being folded outwardly, in example embodiments, the first portion and the second portion may be folded inwardly (i.e., folded radially inward towards the longitudinal axis).

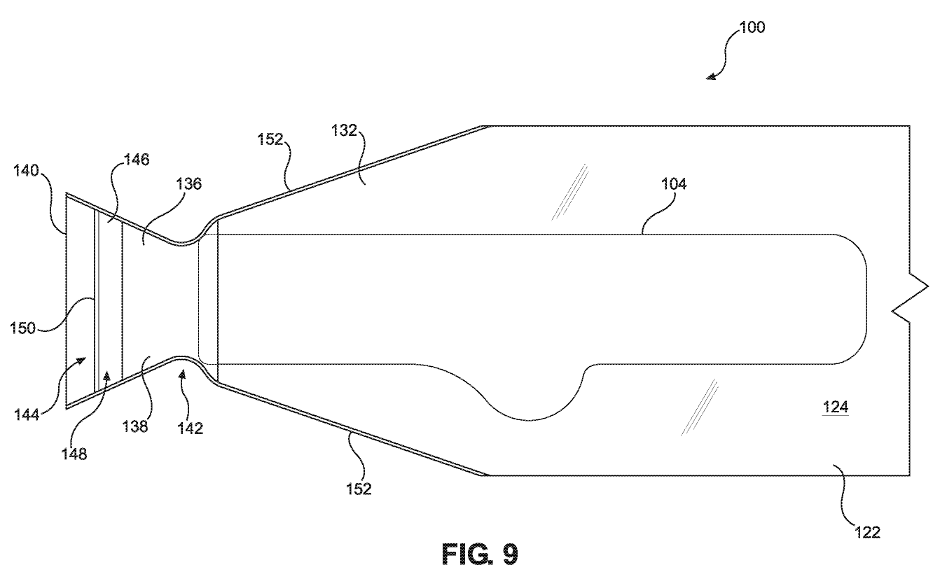

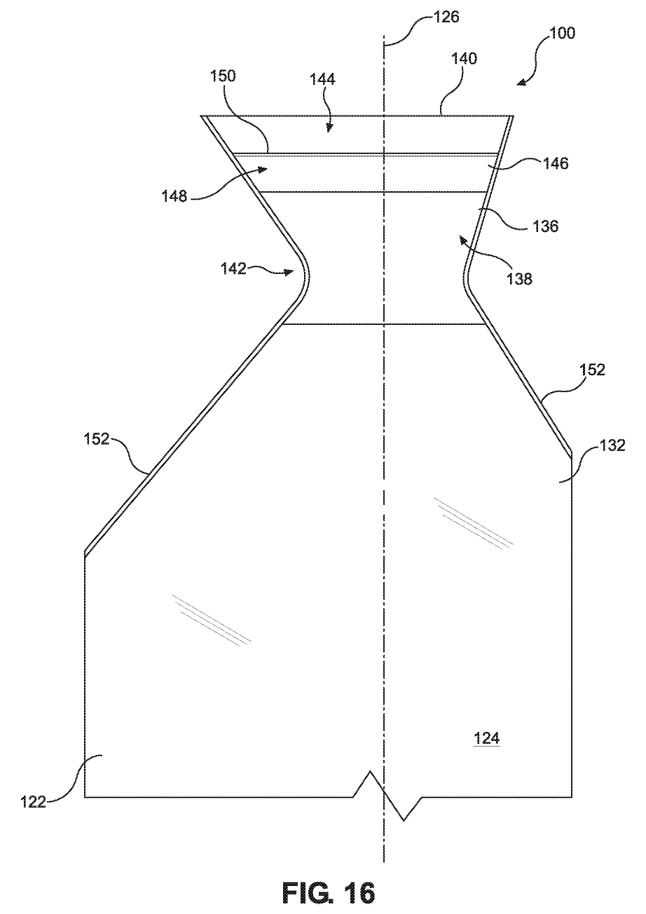

As shown in FIGS. 8-22, the first portion includes lateral sides that symmetrically converge toward longitudinal axis 126, such as shown in FIGS. 8, 10, 12, 14, 18, and 20-22, for example, or asymmetrically converge toward longitudinal axis 126, such as shown in FIG. 16, for example, at a transition area between the first portion and the second portion. Additionally, or alternatively, the first portion and/or the second portion may have any suitable dimensions, e.g., a diameter and/or a length, to accommodate medical devices having varying body dimensions and shapes, as shown in FIGS. 9, 11, 13, 15, 17, and 19, for example. The second portion converges toward longitudinal axis 126 at throttle area 142, forming a sterile field and biased opening 144. Opening 144 extends outwardly (e.g., diverges, etc.) from longitudinal axis 126 and is biased to an open position to provide fluid communication with passage 124 to facilitate coupling and decoupling attachment 106 to body 104 during a surgical procedure, while maintaining the sterile field. The second portion is configured to retain body 104 at attachment plate 108 within throttle area 142 forming the sterile field.

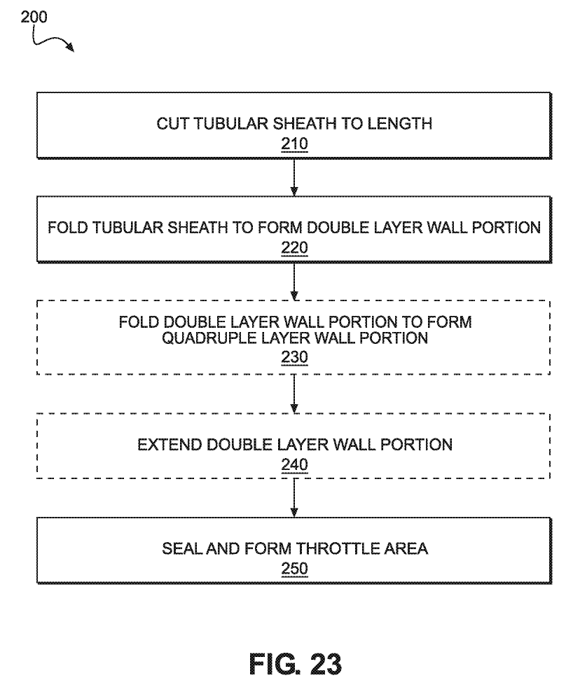

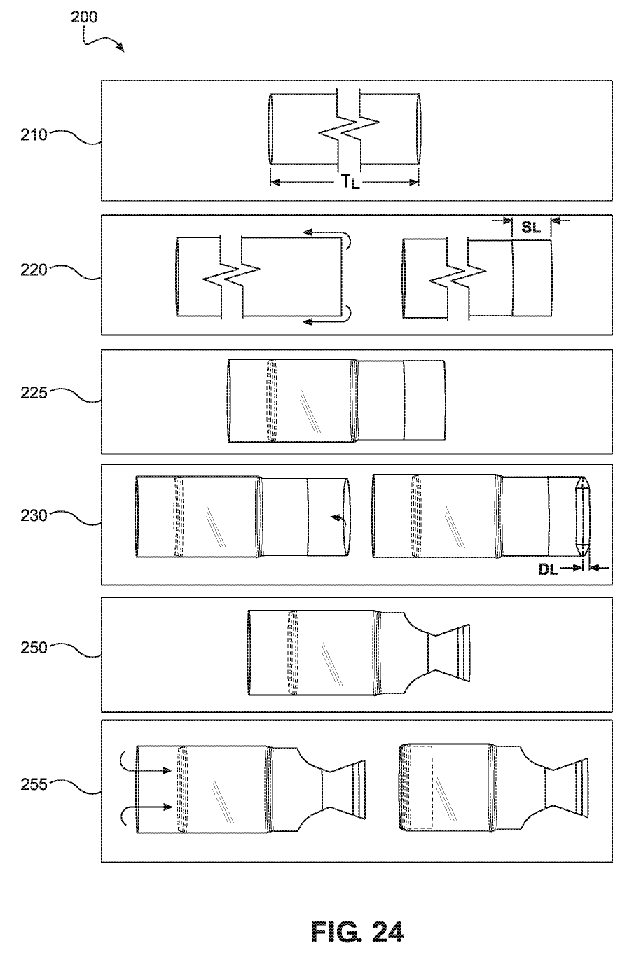

FIGS. 23-24 illustrate an example method 200 for forming (e.g., manufacturing, producing, etc.) a cover assembly, such as cover assembly 100 configured to cover or surround at least a portion of a medical device including, without limitation, medical device 102 having body 104 and attachment 106, such as a removable interventional catheter system or an ablation probe, for example, removably coupled to body 104 at attachment plate 108 positioned at distal end 110 of body 104.

Referring generally to FIGS. 23-24, at 210, a tube stock of a suitable film material (e.g., a polyethylene film material) is cut into a plurality of cover assemblies each including a tubular sheath initially formed of a single layer wall defining a passage along a longitudinal axis extending a length of the tubular sheath between a first end (e.g., a distal end) and an opposing second end (e.g., a proximal end). As shown in FIG. 24, each tubular sheath is initially cut to a suitable length T_{L} (e.g., perpendicular to the longitudinal axis), in particular, a length of 12 inches to 200 inches, and, more particularly, 12 inches to 100 inches. In alternative example embodiments, the length of the tubular sheath is less than 12 inches or greater than 200 inches.

At 220, the first end (e.g., the distal end) of the tubular sheath forming the single layer wall portion is folded outwardly from the longitudinal axis at a first fold line to form a first fold area including a double layer wall portion. As shown in FIG. 24, in example embodiments, the first fold area has a suitable length S_{L} along the longitudinal axis, particularly, a length of 0.5 inches to 8 inches, and, more particularly, 1 inch to 6 inches, and, even more particularly, 2.4 inches. More particularly, in example embodiments, the first fold area has a length of 2 inches distal of a throttle plane defined within the throttle area to 4 inches proximal of the throttle plane. In example embodiments, a template is utilized to guide or facilitate folding of the first end outwardly at the first fold line as indicated on the template to form the first fold area including the double layer wall portion. In example embodiments, at 225, with the distal end of the tubular sheath folded at first fold line to form the first fold area, the proximal end of the tubular sheath is folded 206 a plurality of times in a telefolding process configured to form a cover assembly, as shown in FIGS. 24, for example. In example embodiments, at 220, the first end is folded inwardly, rather than outwardly, from the longitudinal axis at the first fold line to form the first fold area including the double layer wall portion.

At 230, after the telefolding process in 225, with the cover assembly laid flat, at least one portion of the cover assembly, (e.g., a first portion of the cover assembly) within the first fold area is folded outwardly from the longitudinal axis or inwardly toward the longitudinal axis at a second fold line to form a second fold area formed of a quadruple layer wall portion within or adjacent the first fold area, as shown in FIG. 24. In example embodiments, the double layer wall portion is folded at the second fold line adjacent (i.e., proximal to, the first fold line). As shown in FIG. 24, in example embodiments, the second fold area has a suitable length D_{L} along the longitudinal axis, particularly, a length of 0.25 inches to 2.0 inches, and, more particularly, 0.5 inches to 1 inch. In example embodiments, at 240, double layer wall portion at the second side of the cover assembly, opposite the first side of the cover assembly that is folded to form the second fold area, extends in a distal direction along the longitudinal axis with respect to the throttle area to at least partially define the sterile field. In example embodiments, at 230, the double layer wall portion is folded inwardly, rather than outwardly, from the longitudinal axis at the first second fold line to form the first second fold area including the quadruple layer wall portion.

At 250, the distal end of the tubular sheath is sealed using a suitable sealing process. More specifically, the single layer wall portion is sealed, the double layer wall portion is sealed at the first fold area, and the quadruple layer wall portion is sealed at the second fold area to form a throttle area configured to retain a body of a medical device at an attachment plate properly positioned within the passage, as shown in FIG. 24. In example embodiments, each of the single layer wall portion, double layer wall portion, and quadruple layer wall portion converges toward the longitudinal axis at the first end (e.g., the distal end) of the cover assembly. Each of the single layer wall portion, the double layer wall portion, and the quadruple layer wall portion is then sealed using any suitable sealing technique including, without limitation, a heat-sealing process, a thermoforming process, an adhesive process, and/or a chemical sealing process, to form the throttle area as shown, for example, in FIGS. 2-22.

In example embodiments, the first fold area and the second fold area are secured to a template or bracket, using a suitable retention clamp, for example, prior to sealing in 250. In example embodiments, the cover assembly is positioned within a sealing machine and the sealing is initiated using suitable sensors, such as one or more OTB sensors. After the first layer wall portion, the double layer wall portion, and the quadruple layer wall portion are sealed to form the throttle area, excess film material is removed and discarded. In example embodiments, sealing the double layer wall portion at the first fold area to form a throttle area configured to retain the body of the medical device at the attachment plate includes converging the double layer wall portion toward the longitudinal axis within the first fold area to form an opening that is biased to an expanded or open position such that the double layer wall portion diverges from the longitudinal axis of the passage. The opening provides fluid communication with the passage to facilitate coupling the attachment to the body. With the biased opening, a surgeon may more easily remove an attachment and couple a new attachment to the body of the medical device utilizing a single hand while maintaining a sterile environment.

In example embodiments, as shown in FIG. 24, at 255, the proximal end of the cover assembly is folded 216 inwardly toward the longitudinal axis and tucked into the passage formed by the tubular sheath of the cover assembly to facilitate shipping and storage of the cover assembly (e.g., folded into the telescopic fold towards the distal end of the cover assembly). More specifically, the cover assembly in the telefolding configuration is secured to a bracket or template and the proximal end of the cover assembly is folded inwardly toward the longitudinal axis and tucked into the passage formed by the tubular sheath of the cover assembly. In example embodiments, suitable labels, such as labels including arrows to guide proper unfolding of the cover assembly in the surgical environment prior to positioning the cover assembly around at least a portion of the medical device, are adhesively coupled to the cover assembly. In example embodiments, the labels are collinear to an edge of the cover assembly with the cover assembly in the telefolding configuration.

### Embodiments of the disclosure can be described in view of the following clauses:

1A. A cover assembly configured to cover at least a portion of a medical device, the medical device having a body and an attachment removably coupled to the body at an attachment plate positioned at a distal end of the body, the cover assembly comprising:

a film material forming a tubular sheath, the tubular sheath defining a passage configured to receive at least a portion of the medical device, the passage extending along a longitudinal axis between a first end (e.g., a distal end), and an opposing second end (e.g., a proximal end), the tubular sheath comprising a single layer wall portion at the second end of the tubular sheath, the single layer wall portion transitioning into a double layer wall portion at a first fold area, the double layer wall portion defining a throttle area configured to retain the body at the attachment plate.

2A. The cover assembly according to clause 1A, wherein the single layer wall portion transitions into the double layer wall portion at a first fold line.

3A. The cover assembly according to clause 2A, wherein the double layer wall portion transitions into a quadruple layer wall portion at a second fold area adjacent the first fold area at a second fold line proximal to the first fold line.

4A. The cover assembly according to any of clauses 1A-3A, wherein the single layer wall portion converges toward the longitudinal axis at a first end portion (e.g., a distal end portion) of the single layer wall portion.

5A. The cover assembly according to any of clauses 1A-4A, wherein the double layer wall portion converges toward the longitudinal axis within the first fold area to form an opening biased to an open position, the opening providing fluid communication with the passage to facilitate coupling the attachment to the body.

6A. The cover assembly according to clause 1, wherein at least a portion of the double layer wall portion extends distally from the attachment plate to at least partially define a sterile field.

7A. The cover assembly according to any of clauses 1A-6A, wherein the film material comprises a polyethylene film material.

8A. A cover assembly positionable about at least a portion of a medical device, the medical device having a body and an attachment removably coupled to the body at an attachment plate at a distal end of the body, the cover assembly comprising:

a first portion comprising a single layer film material forming a tubular passage along a longitudinal axis of the first portion between a first end and an opposing second end of the first portion;

a second portion formed at the first end of the first portion, the second portion having two layers and configured to surround the distal end of the body; and

a third portion formed adjacent to the second portion, the third portion having four layers.

9A. The cover assembly according to clause 8A, wherein the first portion converges toward the longitudinal axis at a transition area between the first portion and the second portion.

10A. The cover assembly according to clause 8A, wherein the second portion at least partially defines a sterile field and converges toward the longitudinal axis at the sterile field and forms a biased opening extending outwardly from the longitudinal axis, the biased openingin fluid communication with the passage to facilitate coupling the attachment to the body.

11A. The cover assembly according to any of clauses 8A-9A, wherein the second portion at least partially defines a sterile field and is configured to retain the body at the attachment plate within a sterile field.

12A. The cover assembly according to any of clauses 8A-11A, wherein the first portion is folded at a first fold line in a transition area between the first portion and the second portion to form the two layers of the second portion.

13A. The cover assembly according to any of clauses 8A-12A, wherein the second portion is folded at a second fold line to form the four layers of the third portion.

14A. The cover assembly according to any of clauses 8A-13A, wherein the cover assembly comprises a polyethylene film material.

15A. A method for producing a cover assembly configured to cover at least a portion of a medical device, the medical device having a body and an attachment removably coupled to the body at an attachment plate positioned at a distal end of the body, the method comprising:

cutting to a length a film material forming a tubular sheath, the tubular sheath comprising a single layer wall configured to define a passage along a longitudinal axis extending a length of the tubular sheath between a first end and an opposing second end; folding at a first fold line the first end of the tubular sheath to form a first fold area comprising a double layer wall portion; and sealing the double layer wall portion at the first fold area to form a throttle area configured to retain the body of the medical device at the attachment plate.

16A. The method according to clause 15A, further comprising folding at a second fold line at least a portion of the first end of the tubular sheath to form a second fold area comprising a quadruple layer wall portion adjacent to or within the first fold area.

17A. The method according to clause 16A, wherein folding at a second fold line the first end of the tubular sheath to form a second fold area comprising a quadruple layer wall portion comprises folding the double layer wall portion at a second fold line adjacent The first fold line.

18A. The method according to any of clauses 15A-17A, wherein sealing the double layer wall portion at the first fold area to form a throttle area configured to retain the body of the medical device at the attachment plate comprises converging the single layer wall toward the longitudinal axis at the first end of the single layer wall.

19A. The method according to any of clauses 15A-18A, wherein sealing the double layer wall portion at the first fold area to form a throttle area configured to retain the body of the medical device at the attachment plate comprises converging the double layer wall portion toward the longitudinal axis within the first fold area to form an opening biased to an open position, the opening providing fluid communication with the passage to facilitate coupling the attachment to the body.

20A. The method according to any of clauses 15A-19A, further comprising extending the double layer wall portion from the attachment plate to at least partially define a sterile field.

As used herein, an element or step recited in the singular and preceded by the word “a” or “an” should be understood as not necessarily excluding the plural of the elements or steps. Further, references to “one implementation” are not intended to be interpreted as excluding the existence of additional implementations that also incorporate the recited features. Moreover, unless explicitly stated to the contrary, implementations “comprising” or “having” an element or a plurality of elements having a particular property could include additional elements not having that property.

When introducing elements of aspects of the disclosure or the implementations thereof, the articles “a,” “an,” “the,” and “said” are intended to mean that there are one or more of the elements. The terms “comprising,” “including,” and “having” are intended to be inclusive and mean that there could be additional elements other than the listed elements. The terms “implementation” and “embodiment” are intended to mean “an example of.” Unless explicitly stated to the contrary, the phrase “one or more of the following: A, B, and C” means “at least one of A and/or at least one of B and/or at least one of C.”

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

What is claimed is:

1. A cover assembly configured to cover at least a portion of a medical device, the medical device having a body and an attachment removably coupled to the body at an attachment plate positioned at a distal end of the body, the cover assembly comprising:
a film material forming a tubular sheath, the tubular sheath defining a passage configured to receive at least a portion of the medical device, the passage extending along a longitudinal axis of the tubular sheath between a first end and an opposing second end, the tubular sheath comprising a single layer wall portion at the second end of the tubular sheath, the single layer wall portion transitioning into a double layer wall portion at a first fold area, the double layer wall portion defining a throttle area configured to retain the body at the attachment plate.

2. The cover assembly according to claim 1, wherein the single layer wall portion transitions into the double layer wall portion at a first fold line.

3. The cover assembly according to claim 2, wherein the double layer wall portion transitions into a quadruple layer wall portion at a second fold area adjacent the first fold area at a second fold line proximal to the first fold line.

4. The cover assembly according to claim 1, wherein the single layer wall portion converges toward the longitudinal axis at a first end portion of the single layer wall portion.

5. The cover assembly according to claim 1, wherein the double layer wall portion converges toward the longitudinal axis within the first fold area to form an opening biased to an open position, the opening providing fluid communication with the passage to facilitate coupling the attachment to the body.

6. The cover assembly according to claim 1, wherein at least a portion of the double layer wall portion extends distally from the attachment plate to at least partially define a sterile field.

7. The cover assembly according to claim 1, wherein the film material comprises a polyethylene film material.

8. A cover assembly positionable about at least a portion of a medical device, the medical device having a body and an attachment removably coupled to the body at an attachment plate at a distal end of the body, the cover assembly comprising:
a first portion comprising a single layer film material forming a tubular passage along a longitudinal axis of the first portion between a first end and an opposing second end of the first portion;
a second portion formed at the first end of the first portion, the second portion having two layers and configured to surround the distal end of the body; and
a third portion formed adjacent to the second portion, the third portion having four layers.

9. The cover assembly according to claim 8, wherein the first portion converges toward the longitudinal axis at a transition area between the first portion and the second portion.

10. The cover assembly according to claim 8, wherein the second portion at least partially defines a sterile field and converges toward the longitudinal axis at the sterile field and forms a biased opening extending outwardly from the longitudinal axis, the biased opening in fluid communication with the passage to facilitate coupling the attachment to the body.

11. The cover assembly according to claim 8, wherein the second portion at least partially defines a sterile field and is configured to retain the body at the attachment plate within a sterile field.

12. The cover assembly according to claim 8, wherein the first portion is folded at a first fold line in a transition area between the first portion and the second portion to form the two layers of the second portion.

13. The cover assembly according to claim 8, wherein the second portion is folded at a second fold line to form the four layers of the third portion.

14. The cover assembly according to claim 8, wherein the cover assembly comprises a polyethylene film material.

15. A method for producing a cover assembly configured to cover at least a portion of a medical device, the medical device having a body and an attachment removably coupled to the body at an attachment plate positioned at a distal end of the body, the method comprising:
cutting to a length a film material forming a tubular sheath, the tubular sheath comprising a single layer wall configured to define a passage along a longitudinal axis extending a length of the tubular sheath between a first end and an opposing second end;
folding at a first fold line the first end of the tubular sheath to form a first fold area comprising a double layer wall portion; and
sealing the double layer wall portion at the first fold area to form a throttle area configured to retain the body of the medical device at the attachment plate.

16. The method according to claim 15, further comprising folding at a second fold line at least a portion of the first end of the tubular sheath to form a second fold area comprising a quadruple layer wall portion.

17. The method according to claim 16, wherein folding at a second fold line at least a portion of the first end of the tubular sheath to form a second fold area comprising a quadruple layer wall portion comprises folding the double layer wall portion at the second fold line adjacent the first fold line.

18. The method according to claim 15, wherein sealing the double layer wall portion at the first fold area to form a throttle area configured to retain the body of the medical device at the attachment plate comprises converging the single layer wall toward the longitudinal axis at the first end of the single layer wall.

19. The method according to claim 15, wherein sealing the double layer wall portion at the first fold area to form a throttle area configured to retain the body of the medical device at the attachment plate comprises converging the double layer wall portion toward the longitudinal axis within the first fold area to form an opening biased to an open position, the opening providing fluid communication with the passage to facilitate coupling the attachment to the body.

20. The method according to claim 15, further comprising extending the double layer wall portion from the attachment plate to at least partially define a sterile field.
